(19) United States
(12) Patent Application Publication
(10) Pub. No.: US 2007/0148140 A1
(43) Pub. Date: Jun. 28, 2007

(54) COMPOSITIONS AND METHODS TO ENHANCE VIABILITY AND FUNCTION OF ISLET CELLS

(75) Inventor: Zoltan Kiss, Austin, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: ZOLTAN LABORATORIES LLC, Austin, MN (US)

(21) Appl. No.: 11/614,176

(22) Filed: Dec. 21, 2006

Related U.S. Application Data
(60) Provisional application No. 60/754,524, filed on Dec. 28, 2005.
(60) Provisional application No. 60/754,412, filed on Dec. 28, 2005.

(51) Int. Cl. A61K 38/46; A61K 35/37

(52) U.S. Cl. 424/93.7; 424/94.6

(57) ABSTRACT

This invention uses placental alkaline phosphatase (“PALP”), and other members of the alkaline phosphatase family, to reduce the death and thereby maintain or enhance the viability and function of insulin-producing islet β-cells including insulin secretion. PALP may be administered to a patient that has received transplanted islet cells to protect the transplanted islets against ROS-mediated attacks by the patient's immune system. Transferrin and other promoters of islet survival may also be used to enhance the effects of PALP on islet viability both in vivo and in vitro.

### CLAIM OF PRIORITY

This application claims the benefit of provisional applications Ser. No. 60/754,524, filed Dec. 28, 2005, entitled “Compositions and methods for protecting pancreatic islets”; and Ser. No. 60/754,412, filed Dec. 28, 2005, entitled “Compositions and methods for stimulating insulin secretion from islets,” each of which is incorporated by reference.

### FIELD OF THE INVENTION

The present invention generally involves the use of placental alkaline phosphatase, either alone or in combination with transferrin (TF) or known promoters of islet survival and/or insulin secretion, to reduce the death of islet β-cells and thereby maintain or enhance their viability in vitro and in vivo. The invention also involves the use of placental alkaline phosphate alone and particularly in combination with transferrin (TF) to increase secretion of insulin from human islets.

### BACKGROUND

Despite large variations in carbohydrate intake with various meals, blood glucose normally remains in a narrow range between 4 and 6 mM in non-diabetic individuals. Such tight control is regulated by the balance among three major mechanisms, i.e. (i) glucose absorption from the intestine, (ii) glucose production by the liver, and (iii) uptake and metabolism of glucose by the peripheral tissues, mainly the skeletal muscle and fat tissue. In skeletal muscle and fat tissue, insulin increases the uptake of glucose, increases the conversion of glucose to glycogen, and increases conversion of glucose to fat (mainly triglycerides). In the liver, insulin inhibits the release of glucose from glycogen. Insulin, produced by islet β-cells in the pancreas, is the only known hormone which can regulate all three mechanisms required to maintain the blood glucose level in the normal range [Saltiel, A. R. and Kahn, C. R., “Insulin signaling and the regulation of glucose and lipid metabolism.” Nature 414, 799-806 (2001)].

Extensive destruction of insulin-producing islet β-cells in the pancreas is the hallmark of type 1 diabetes. However, major loss of islet cells also frequently occurs in aging and diseased patients as well as more severe cases of type 2 diabetic subjects. The consequence of reduced secretion of insulin into the blood is elevated blood glucose level. In turn, higher than normal levels of glucose in the blood accelerate the destruction of the remaining islet cells. The destructive effects of high glucose are mediated by reactive oxygen species (ROS) often involving pro-apoptotic cytokines [Tabatabaie T., Vasquez-Weldon, A., Moore, D. R. and Kotake, Y. (2003), “Free radicals and the pathogenesis of type 1 diabetes: β-cell cytokine-mediated free radical generation via cyclooxygenase-2,” Diabetes, 52, 1994-1999; Roberston, R. P., Harmon, J., Tran, P. O., Tanaka, Y. and Takahashi, H. “Glucose toxicity in β-cells: Type 2 diabetes, good radicals gone bad, and the glutathione connection,” (2003) Diabetes, 52, 581-587]. The result is further reduction of insulin and therefore still higher levels of glucose in the patient's blood. Dangerously high levels of glucose in the blood, or hyperglycemia, may lead to diabetes [Mandrup-Poulsen, T. (2001) “β-cell apoptosis,” Diabetes, 50, (Suppl. 1): S58-S63].

Higher levels of saturated free fatty acids in the circulation may also lead to islet cell dysfunction and destruction via apoptosis [Kwon, G., Pappan, K. L., Marshall, C. A., Schaffer, J. E. and McDaniel, M. L. (2004) “cAMP dose-dependently prevents palmitate-induced apoptosis by both protein kinase A- and cAMP-guanine exchange factor-dependent pathways in β-cells,” J. Biol. Chem., 279, 8938-8945]. In contrast, unsaturated fatty acids have exhibited some protective effects [Eitel, K., Staiger, H., Brendel, M. D., Brandhorst, D., Bretzel, R. G., Haring, H. U. and Kellerer, M. (2002) “Different role of saturated and unsaturated fatty acids in β-cell apoptosis,” Biochim. Biophys. Res. Commun., 299, 853-856; Maedler, K., Oberholzer, J., Bucher, P., Spinas, G. A. and Donath, M. Y. (2003) “Monounsaturated fatty acids prevent the deleterious effects of palmitate and high glucose on human pancreatic β-cell turnover and function.” Diabetes, 52, 726-733].

To reduce hyperglycemia resulting from loss of islet cells, and thus prevent further islet cell death, diabetic patients are presently treated with insulin and/or other anti-diabetic agents. In severe cases of hyperglycemia, when a patient's islet cells are destroyed so extensively that survival requires frequent administration of insulin, islet cells may be transplanted into the patient. However, short supply of islet cell donors and inactivation of islet functions during the isolation process and following transplantation seriously limits this form of therapy [Ryan, E. A., Paty, B. W., Senior, P. A., Bigam, D., Alfadhli, E., Kneteman, N. M., Lakey, J. R. T. and Shapiro, A. M. J. (2005) “Five-year follow-up after clinical islet transplantation,” Diabetes, 54, 2060-2069].

Since oxidative injury partly accounts for the impairment of islet function, treatment with an antioxidant may improve the yield and the function of transplanted islets in vitro [Bottino, R., Balamurugan, A. N., Tse, H., Thirunavukkarasu, C., Ge, X., Profozich, J., Milton, M., Ziegenfuss, A., Trucco, M. and Piganelli, J. D. (2004) “Response of human islets to isolation stress and the effect of antioxidant treatment,” Diabetes, 53, 2559-2568; Mysore, T. B., Shinkel, T. A., Collins, J., Salvaris, E. J., Fisicaro, N., Murray-Segal, L. J., Johnson, L. E. A., Lepore, D. A., Walters, S. N., Stokes, R., Chandra, A. P., O'Connell, P. J., d'Apice, A. J. F. and Cowan, P. J. (2005) “Overexpression of glutathione peroxidase with two isoforms of superoxide dismutase protects mouse islets from oxidative injury and improves islet graft function,” Diabetes, 54, 2109-2116]. However, there is currently no known clinical method of protecting against either oxidant- or fatty acid-induced β-cell death. A protective agent or combination of protective agents would not only make the islet transplantation method more efficient, but would also improve the function of remaining islets in diabetic subjects who cannot receive islet transplantation. Such protective agent then would be expected to maintain or enhance, via increased viability of β-cells, the synthesis and secretion of insulin.

### SUMMARY OF THE INVENTION

Embodiments of the invention relate to the use of placental alkaline phosphatase (“PALP”), alone or in combination with other members of the alkaline phosphatase family, alone or in combination with transferrin, to enhance viability of insulin producing β-cells and thereby insulin secretion.

As a primary consequence of increased viability of β-cells, PALP also enhances the amount of insulin secreted from human islets. Both aspects of PALP actions on islet β-cells, i.e. increased viability and insulin secretion, are enhanced by transferrin (TF). Other agents that are known to enhance the viability of islet β-cells and insulin secretion may also be employed to further improve these effects of PALP or the combination of PALP and TF.

The term “maintaining viability” means that placental alkaline phosphatase and transferrin maintains the number of viable cells under conditions that otherwise induce the death of islet cells. The term “enhancing viability” means that placental alkaline phosphatase and transferrin increase the number of viable islet cells. Both “maintenance” and “enhancement” of islet cell viability results from the ability of placental alkaline phosphatase and transferrin to reduce or prevent the death of these cells. It is assumed that increased viability enhances the capacity of islet cells to increase insulin release.

Although the term placental alkaline phosphatase (PALP) is used throughout the application, other members of the human alkaline phosphatase family may be used instead of PALP. The ability of PALP to enhance the viability of insulin producing islet β-cells that eventually leads to increased insulin secretion has many therapeutic applications. For example, it may be used alone or along with TF and other protective agents to protect islets in vitro against isolation stress, largely mediated by ROS. PALP may also be administered, alone or along with TF and other protective agents, to a patient that received transplanted islet cells to protect the transplanted islets against ROS-mediated attacks by the patient's immune system. PALP may also be used, alone or along with TF and other protective agents, to treat type 1 or type 2 diabetic patients to reduce the death of the patient's islets by factors such as ROS and saturated fatty acids. The overall result of in vivo protective effects of PALP, exerted alone or together with TF or other protective agents, is increased insulin secretion and better control of blood glucose level.

In one embodiment, the invention provides a method to reduce or prevent cell death thereby maintaining or increasing the viability of mammalian islet cells in vitro, comprising contacting the islet cells with an effective amount of an alkaline phosphatase or an active derivative thereof in the absence or presence of TF or another promoter of islet cell survival.

In another embodiment, the invention provides a method to enhance the viability of islet cells and thereby promoting insulin secretion in a mammal by administering an alkaline phosphatase or an active derivative thereof alone or together with TF or another promoter of islet cell survival to the mammal. In this embodiment the islet cells may be transplanted into the mammal.

In yet another embodiment, the invention provides media for the isolation and storage of islet cells including 1 to 100 μg per ml of alkaline phosphatase or an active derivative. Effective amounts of TF (1 to 50 μg per ml) or another promoter of islet cell survival may be added to these alkaline phosphatase-containing media.

In a further embodiment, the invention provides a treatment regimen for the treatment of a mammal with type 1 or type 2 diabetes comprising periodically administering a therapeutically effective amount of an alkaline phosphatase or an active derivative thereof, alone or together with TF or another promoter of islet cell survival.

In still another embodiment, the invention provides for the use of alkaline phosphatase or an active derivative thereof alone or together with TF in the manufacture of a composition useful for the enhancement of viability islet cells as well as insulin secretion in vivo.

In yet another embodiment, the invention provides for the use of alkaline phosphatase or an active derivative thereof alone or together with TF in the manufacture of a composition useful for the enhancement of viability of islet cells in vitro.

In some embodiments, the mammal is administered a therapeutically effective amount of an alkaline phosphatase alone or together with a therapeutically effective amount of TF. The term “therapeutically effective amount” of PALP is used in this application to mean a dose that is effective in enhancing the viability of islet cells as well as insulin secretion. The term “therapeutically effective amount” of TF is used in this application to mean a dose that is effective in increasing the effects of PALP on the viability of islet cells as well as insulin secretion.

As used herein, the terms “PALP and TF” and the phrases “human PALP and human TF” are used interchangeably to refer to placental alkaline phosphatase and transferrin, respectively. The terms “active PALP and active TF” are used in this application to refer to the human proteins and their glycosylated and non-glycosylated forms as well as peptides derived from these proteins. The terms “active PALP and active TF” also include recombinant forms of these proteins as far as they reproduce the effects of the corresponding natural proteins isolated from human tissues (placenta or blood). The term “substantially purified” is used herein to encompass preparations of PALP and TF that are obtained from raw extracts by one or more purification steps, such as, for example, solvent extraction, column chromatography separation, or other separation methods. These methods may enrich the concentration of these proteins, relative to the raw extract, to an extent that they become the major, but not necessarily a dominant, component. Substantially purified PALP and TF preparations can be used as far as the remaining components do not pose any significant health risk and do not reduce their beneficial effects. The commercial PALP preparation used for some of the experiments in the present invention is a substantially purified preparation. The term “highly purified” is used herein to encompass preparations of PALP and TF in which PALP or TF is the dominant component representing at least 95% of the total protein content. The term “highly purified” should not be construed to connote absolute purity. The commercial TF preparation used for this invention was highly purified.

When used with respect to a protein preparation, the phrase “homogeneous” refers to a protein preparation where the protein of interest is the only protein that can be clearly detected (such as by using coomassie blue or silver stain for protein staining) by SDS-PAGE gel electrophoresis. This definition allows for the detection of multiple bands represented by various forms of the same protein (e.g., glycosylated or phosphorylated forms), so long as the proteins in the separate bands have the same amino acid composition. By way of example, a homogeneous PALP preparation used in the experiments described herein contained only one band by SDS-PAGE gel electrophoresis.

### BRIEF DESCRIPTION OF THE DRAWINGS

FIG. 1 is a digital image of a gel separation, demonstrating that the PALP used in the examples was homogeneous or nearly homogeneous, except when indicated otherwise.

FIG. 2 is a digital image demonstrating that incubation of T47D cells in serum-free medium from day 0 (A) until day 6 (B) results in the loss of about 20% of cells. In the presence of 5 μg/ml purified PALP (C) or 15 μg/ml purified PALP (D) there are further decreases (about 15-30%) in cell number.

### DETAILED DESCRIPTION OF THE INVENTION

The present invention uses placental alkaline phosphatase, either alone or in combination with transferrin (TF) or other promoters of islet survival and insulin secretion, to reduce or prevent the death and thereby maintain or enhance viability of islet β-cells and as a consequence maintain or enhance insulin secretion both in vivo and in vitro. Utility of the invention includes reducing the death and thereby maintaining islet viability during islet isolation for transplantation, maintaining viability and functionality of islets after transplantation, and maintaining viability and functionality of islets in diseased subjects when the disease state is associated with declining number and/or function of islets.

Active Components

Placental Alkaline Phosphatase (PALP)

PALP is one of the presently known four members of the human alkaline phosphatase enzyme family that hydrolyzes phosphate-containing compounds at alkaline pH [J. L. Millan, and W. H. Fishman (1995), “Biology of human alkaline phosphatases with special reference to cancer,” Critical Reviews in Clinical Sciences, 32, 1-39]. Other members of this human alkaline phosphatase group that also hydrolyze phosphate-containing compounds at alkaline pH include the tissue non-specific (liver/bone/kidney), the intestinal, and PALP-like (germ cell) alkaline phosphatases. Mature PALP is a dimer of two identical glycosylated subunits. Each subunit has an approximate molecular weight of 66 kDa, as determined by gel electrophoresis.

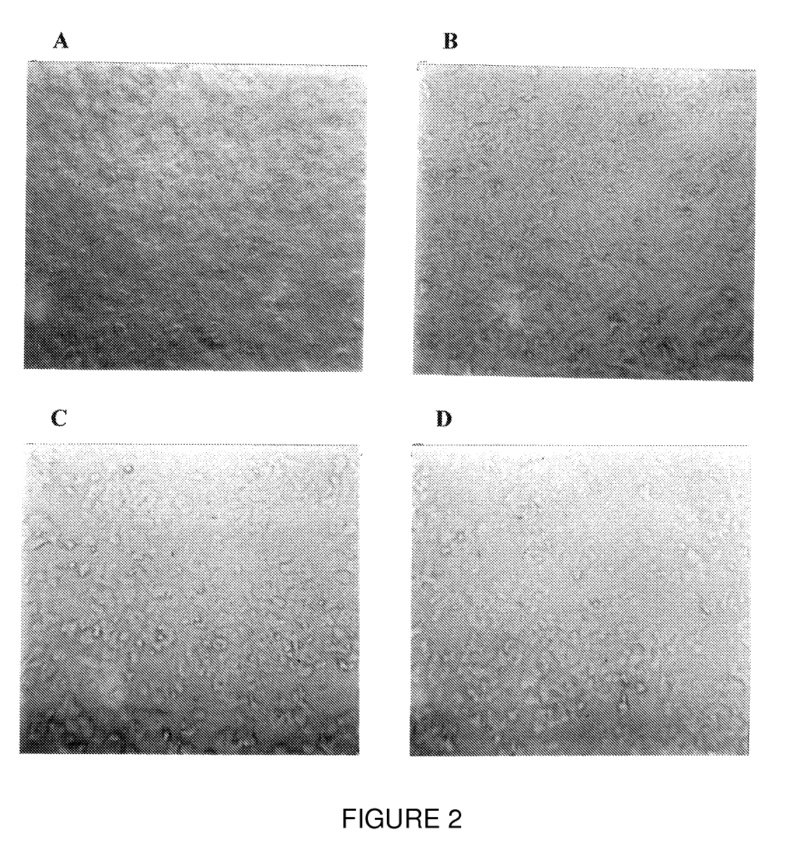

In cell cultures of certain normal cell lines, PALP can promote cell survival and proliferation. For example, PALP was reported to enhance both the proliferation and survival of mouse embryo fibroblasts and fibroblast-like cells derived from the lung of human fetus [Q. -B. She, J. J. Mukherjee, J. -S. Huang, K. S. Crilly, and Z. Kiss (2000), “Growth factor-like effects of placental alkaline phosphatase in human fetus and mouse embryo fibroblasts,” FEBS Letters, 468, 163-167; Q. -B. She, J. J. Mukherjee, T. Chung, and Z. Kiss (2000), “Placental alkaline phosphatase, insulin, and adenine nucleotides or adenosine synergistically promote long-term survival of serum-starved mouse embryo and human fetus fibroblasts,” Cellular Signaling, 12, 659-665]. PALP may also enhance proliferation of human skin fibroblasts and keratinocytes. However, in some other cases PALP reduces cell viability. For example, as it will be illustrated in the Examples with T47D cells (FIG. 2), PALP actually decreases viability of these cells. In yet other cases, PALP does not have a well detectable effect on cell survival. For example, PALP failed to enhance survival (in serum-free medium) of 3A-sub E cells derived from human placenta, Caov-3 human adenocarcinoma cells derived from the ovary, and Hep G2 human hepatocellular carcinoma cells.

In some embodiments, an active PALP derivative that is a smaller fragment of a PALP amino acid sequence and demonstrates efficacy similar to that of native PALP may be used. For example an active derivative may be formed by exchanging amino acids at critical sites, by modifying a PALP amino acid sequence or a sequence of smaller PALP peptides. Likewise, chemical or enzymatic changes in the level and position of glycosylation may maintain or enhance the effects of PALP or its derivatives. In the practice of the present invention, modified PALP, smaller PALP-derived peptides, or modified PALP-derived peptides may be similarly effective or even more effective than native PALP. Each of these is considered to be active derivatives. In the methods of the present invention native glycosylated PALP and its active derivatives as well as non-glycosylated PALP and its active derivatives may be used. It has been demonstrated that alkaline phosphatase activity is not required in native PALP to stimulate mitogenesis in fibroblasts. For example, both digestion of PALP with the protease bromelain and elimination of alkaline phosphatase activity through mutation provided an active derivative with respect to stimulation of skin cell proliferation [U. S. patent application Ser. No. 10/653,622, filed Sep. 2, 2003, and entitled “Use of Placental Alkaline Phosphatase to Promote Skin Cell Proliferation” (Pub. No. US 2005/0048046 A1, published March 3, 2005), incorporated by reference herein.

Transferrin (TF)

In embodiments of the present invention, TF may be used in combination with PALP to enhance the production and secretion of insulin by islet cells. TF is a glycoprotein with an approximate molecular weight of 80 kDa. One of its functions is to carry iron from the sites of intake into the systemic circulation and then to the cells and tissues. TF is also known to enhance proliferation of several cell types and, therefore, it is present in most commercial growth media. It has been less known how TF affects cell survival particularly in case of islet cells. The basic properties of TF and the receptor through which TF enters the cells have been studied [Qian, Z. M., Li, H., Sun, H. and Ho, K. (2002), “Targeted drug delivery via the transferrin receptor-mediated endocytosis pathway,” Pharmacol Rev., 54, 561-587]. TF enhances the promoting effects of PALP on both islet cell viability and insulin secretion.

TF is commercially available in both iron-free and iron-containing species. In some embodiments, partially iron-saturated TF was used; in other embodiments, iron-free preparations were used.

Promoters of Actions of PALP and TF on Enhancing Islet Cell Survival and Insulin Secretion

Additional promoters of the actions of PALP and TF on islet survival and insulin secretion include growth hormone and prolactin [Nielsen, J. H., Galsgaard, E. D., Moldrup, A., Friedrichsen, B. N., Billestrup, N., Hansen, J. A. Lee, Y. C. and Carlsson, C. (2001), “Regulation of β-cell mass by hormones and growth factors,” Diabetes, 50 (Suppl. 1): S25-S29], insulin-like growth factor-1 [Lingom, M. K., Dickson, L. M., McCuaig, J. F., Hugi, S. R., Twardzik, D. R. and Rhodes, C. J. (2002) “Activation of IRS-2-mediated signal transduction by IGF-1, but not TGF or EGF, augments pancreatic β-cell proliferation,” Diabetes, 51, 966-976], glucagon-like peptide-1 and its long-acting derivative NN2211 [De Leon, D. D., Deng, S., Madani, R., Ahima, R. S., Drucker, D. J. and Stoffers, D. A. (2003), “Role of endogenous glucagon-like peptide-1 in islet regeneration after pancreatectomy,” Diabetes, 52, 365-371; Rolin, B., Larsen, M. O., Gotfredsen, C. F., Deacon, C. F., Carr, R. D., Wilken, M. and Knudsen, L. B. (2002), “The long-acting GLP-1 derivative NN2211 ameliorates glycemia and increases β-cell mass in diabetic mice,” Am. J. Physiol. Endocrinol. Metab., 283, E745-E752], parathyroid hormone-related protein [Cebrian, A., Garcia-Ocana, A., Takane, K. K., Sipula, D., Stewart, A. F. and Vasavada, R. C. (2002), “Overexpression of parathyroid hormone-related protein inhibits pancreatic β-cell death in vivo and in vitro,” Diabetes, 51, 3003-3013], gastrin [Rooman, I., Lardon, J. and Bouwens, L. (2002), “Gastrin stimulates β-cell neogenesis and increases islet mass from transdifferentiated but not from normal exocrine pancreas tissue,” Diabetes, 51, 686-690], thiazolidinediones such as pioglitazone [Ishida, H., Takizawa, M., Ozawa, S., Nakamichi, Y., Yamaguchi, S., Katsuta, H., Tanaka, T., Maruyama, M., Katahira, H., Yoshimoto, K., Itagaki, E. and Nagamatsu, S. (2004), “Pioglitazone improves insulin secretory capacity and prevents the loss of β-cell mass in obese db/db mice: Possible protection of β-cells from oxidative stress,” Metabolism, 52, 488-494], and α1-antitrypsin [Lawis, E. C., Shapiro, L., Bowers, O. J. and Dinarello, C. A. (2005), “α1-antitrypsin monotherapy prolongs islet allograft survival in mice,” Proc. Natl. Acad. Sci., USA 102, 12153-12158].

In addition, carbon monoxide and antioxidant compounds were also shown to protect islet cells [Gunther, L., Berberat, P. O., Haga, M., Brouard, S., Smith, R. N., Soares, M. P., Bach, F. H. and Tobiasch, E. (2002), “Carbon monoxide protects pancreatic β-cells from apoptosis and improves islet function/survival after transplantation,” Diabetes, 51, 994-999; Bottino, R., Balamurugan, A. N., Bertera, S., Pietropaolo, M., Trucco, M. and Piganelli, J. D. (2002), “Preservation of human islet cell functional mass by anti-oxidative action of a novel SOD mimic compound,” Diabetes, 51, 2561-2567].

All these promoters of islet cell viability and insulin secretion may be used in combination with PALP, or PALP plus TF, in embodiments of the present invention.

Use of Commercial PALP Preparation.

Commercial PALP, purchased from Sigma-Aldrich Inc. contains about 10% PALP, 12% TF, 30% α₁-antitrypsin (AT), 35% albumin, and 13% TF derivatives (proteolytic degradation products of TF) as determined by gel electrophoresis and subsequent sequence analysis. As it will be shown in the Examples, commercial PALP is as effective as purified PALP plus TF both in promoting islet cell viability and insulin secretion from human islets in vitro. Thus, any commercial PALP preparation wherein either PALP or both PALP and TF are present as major proteins (representing more than 5% of total protein) may be used in the invention for enhancing islet cell viability both in vitro and in vivo as far as such preparation is effective and safe.

Compositions of the Active Components

Compositions of PALP for use in embodiments of the present invention may be prepared using a variety of methods. As mentioned above, human PALP is commercially available from Sigma-Aldrich (St. Louis, Mo.; catalog number P3895; CAS Registry Number 9001-78-9) and also from Calbiochem (San Diego, Calif.; catalog number 524604); both preparations may be used without further purification. These commercially available preparations may also be purified using known purification methods before they are administered to a patient.

Recombinant methods of obtaining suitable preparations of PALP or active derivatives are also suitable. Using the cDNA of PALP, recombinant protein may be produced by one of the many known methods for recombinant protein expression. PALP has been cloned and expressed in cells as described by Kozlenkov, et al. [Kozlenkov, A., Manes, T., Hoylaerts, M. F. and Millan, J. L. (2002), “Function assignment to conserved residues in mammalian alkaline phosphatases,” J. Biol. Chem., 277, 22992-22999]. Production of recombinant PALP by bacteria [Beck, R. and Burtscher, H. (1994), “Expression of human placental alkaline phosphatase in Escherichia coli,” Protein Expression and Purification, 5, 192-197] and yeast [Heimo, H., Palmu, K. and Suominen, I. (1998), “Human placenta alkaline phosphatase: Expression in Pichia pastoris, purification and characterization of the enzyme,” Protein Expression and Purification, 12, 85-92] are also suitable methods of obtaining PALP for use in various embodiments of the present invention.

A suitable composition of PALP, and particularly a smaller active derivative, may also be obtained by chemical synthesis using conventional methods. For example, solid-phase synthesis techniques may be used to obtain PALP or an active derivative.

A composition of human PALP may also be obtained by extraction from placental tissue. Human placenta synthesizes the enzyme during pregnancy so that toward the end of the third term, the level of PALP in the placenta tissue and the maternal and fetal blood becomes very high. Therefore, a composition of PALP may be obtained by extraction, such as butanol extraction, of homogenized placenta. Butanol extraction inactivates most of the other placental proteins, including growth factors, but does not reduce the mitogenic or enzymatic activity of PALP. Other methods of extraction from placental tissue are also suitable.

As is the case with the commercially available human PALP, it may be suitable to purify the raw placental extracts of PALP before using it in embodiments of the present invention. Raw placental extracts may contain other proteins, lipids, proteolipids, carbohydrates, metals, vitamins, and the like that may cause unexpected side effects when administered to a patient. Furthermore, in some raw extracts of PALP, the relative concentration of PALP may be too low to result in an increase in the production and secretion of insulin by islets. Substantially purified preparations of PALP may have a much higher concentration of the active component than found in a raw tissue extract. Therefore, it may be suitable to utilize a substantially purified preparation of PALP for treatment in vivo to ensure the quality of the preparation and to exclude the health risks caused by unidentified contaminants. Substantially purified preparations of bone-specific, tissue non-specific, and PALP-like (germ cell) alkaline phosphatase enzymes are all available commercially (for example, from Sigma-Aldrich).

Preparations of TF for use in embodiments of the invention may be obtained through a variety of methods. In one embodiment, commercial TF may be used. For example, Sigma-Aldrich produces three suitable preparations of TF. These include: (i) an essentially iron-free human Apo-TF (aTF; catalog number T 1147 according to the 2004/2005 Sigma Catalog), (ii) an iron-containing (iron content: 1100-1600 μg per 1-g protein) human holo-TF (hTF; catalog number T 4132), and (iii) another low iron-containing (300-600 μg per 1-g protein) essentially endotoxin-free human TF (efTF; catalogue number, T 3309). The TF protein content in these commercially available preparations maybe greater than 97% in aTF and hTF and greater than 98% in effF. Commercial TF preparations may also be further purified using one or more chromatographic steps to obtain a homogeneous TF preparations. However, TF preparations with some impurities may also be used, so long as the given composition includes therapeutically effective amount of TF, and the impurities do not interfere with the beneficial effects of TF.

In another embodiment, TF is obtained from a raw extract of placental tissue. Since TF is a major blood protein, and placental tissue contains a significant volume of blood, human TF may be obtained by extraction from placental tissue. One example of a suitable extraction method is a butanol extraction. Other methods of extraction from placental tissue are also suitable.

Raw extracts of blood or placenta may also be enriched using physical concentration methods in order to create a suitable preparation of TF. The concentration of TF in some raw extracts may be too low to have a blood glucose-lowering effect when administered to a patient. Therefore, raw blood or placenta-derived TF extractions may be treated with one or more purification steps, such as solvent extraction, column chromatography separation, or other separation methods to increase the concentration of TF as compared to the concentration of TF in the raw extract.

The raw extracts of TF may also be treated to purify and remove impurities from the extract. An advantage of using a purified or homogenous preparation of TF in the methods of the present invention is that possible side effects caused by contaminating proteins may be avoided. However, as mentioned above, impure TF may also be used in the methods of the present invention, so long as the composition includes therapeutically effective amount of TF and the impurities do not interfere with the beneficial effects of TF. Since every consecutive purification step results in some loss of the protein, using a TF preparation that is less than homogeneous in the present invention may be more cost-effective.

In another embodiment, TF may be expressed using recombinant methods. In this embodiment, the cDNA of original TF or its point and deletion mutants is expressed in any suitable cell line, for example in insect cells [Tomiya, N., Howe, D., Aumiller, J. J., Pathak, M., Park, J., Palter, K. B., Jarvis, D. L., Betenbaugh, M. J. and Lee, Y. C. (2003), “Complex-type biantennary N-glycans of recombinant human transferrin from Trichoplusia in cells expressing mammalian β-1,4-galactotransferase and β-1,4-N-acetylglucosaminenyltransferase II,” Glycobiology, 13, 23-34]. These and similar techniques may be used to generate, at larger scale, various active recombinant forms of TF and its active derivatives.

A step of heat-activation may be included during preparation of the compositions of PALP. The stimulatory effects of PALP on fibroblast proliferation in vitro may be enhanced by pre-heating it at 65-75° C. for 30 min. [Q. -B. She, J. J. Mukherjee, J. -S. Huang, K. S. Crilly, and Z. Kiss (2000), “Growth factor-like effects of placental alkaline phosphatase in human fetus and mouse embryo fibroblasts.” FEBS Letters, 468, 163-167]. Thus, a step of heat-activation may be included during the final preparation of PALP for injection. Furthermore, the pre-heating of TF at 65-75° C. for 30 min. does not alter its protecting effect on islet cell survival in vitro. Thus, pre-heating of PALP and TF-containing compositions to enhance the effect of PALP is unlikely to alter the effects of TF.

Methods of Use

In one embodiment, PALP is used to reduce the death of mammalian islet cells in vivo by administering a preparation of PALP or an active derivative thereof with or without TF to a mammal, particularly a human. A preparation of PALP or PALP plus TF suitable for use in embodiments of the present invention may be administered in a variety of manners. In one embodiment, the preparation is administered by injection. Any suitable method of injections, such as intravenous, intraarterial, intramuscular, intraperitoneal, intradermal, intraportal or subcutaneous may be used. PALP or PALP plus TF may also be directly injected into the pancreas either alone or together with transplanted islet cells. In another method, osmotic minipumps or any other types of pumps that can be inserted under the skin and provide for controlled protein release are also suitable.

In yet another embodiment, PALP or a mixture of PALP and TF may be prepared as a dry powder and administered, similar to certain solid insulin products such as Exubera [White, S., Bennett, D. B., Cheu, S., Conley, P. W., Guzek, D. B., Gray, S., Howard, J., Malcolmson, R., Parker, J. M., Roberts, P., Sadrzadeh, N., Schumacher, J. D., Seshadri, S., Sluggett, G. W., Stevenson, C. L. and Harper, N. J. (2005), “EXUBERA: Pharmaceutical development of a novel product for pulmonary delivery of insulin,” Diabetes Technol. Ther., 7, 896-906], via inhalation using a suitable inhalation device. The technology required for the production of an inhalation protein preparation, including steps such as chemical stabilization of the protein, dry powder formulation, powder filling and packaging, and a mechanical device for powder dispersal and reliable dosing, is available. However, considering the relatively long half-life time of TF in the circulation (˜5-7 days) which allows once or twice a weekly application, the most common delivery method in the practice of the invention is likely to be by injection.

For injection, alkaline phosphatase preparations (with or without TF) may be dispersed in any physiologically acceptable carrier that does not cause an undesirable physiological effect and is capable of ensuring proper distribution of the protein(s) into the transplanted islet area. Examples of suitable carriers include physiological saline and phosphate-buffered saline. The injectable solution may be prepared by dissolving or dispersing a suitable preparation of the active protein component in the carrier using conventional methods. As examples only, in one embodiment, a suitable composition for the methods of the present invention includes an alkaline phosphatase in a 0.9% physiological salt solution to yield a total protein concentration of 10 mg/ml. In another embodiment, the composition includes an alkaline phosphatase and TF in a 0.9% physiological salt solution to yield a total protein concentration of 20 mg/ml. In yet another embodiment, the composition includes an alkaline phosphatase, TF and another islet survival-promoting agent in a 0.9% physiological salt solution to yield a total protein concentration of 100 mg/ml. PALP, TF and other promoters of islet viability and insulin secretion may also be enclosed in liposomes such as immunoliposomes, or other delivery systems or formulations.

A suitable dosage for systemic administration may be calculated in grams of PALP per square meter of body surface area of the mammal. In one embodiment, the therapeutically effective amount of PALP is between about 0.01-2.5 g of alkaline phosphatase per m² body surface of the mammal, particularly a human. In another embodiment, the therapeutically effective amount of PALP is between 0. 1-1 g per m2 body surface of the mammal. If the method or treatment regiment includes administering TF in combination with PALP, the TF may be administered in a dosage similar to that of PALP. For example, if TF is administered in combination with PALP, the dose may be about 0.01-2.5 g alkaline phosphatase with about 0.01-2.5 g TF per m² body surface of the mammal.

As stated above, a therapeutically effective amount of PALP for use in the treatments is a dose of PALP, either alone or in combination with TF or another promoter of islet survival, that is effective in enhancing viability of islet cells and insulin secretion. In one embodiment, increased viability of islet cells and insulin secretion in vivo may be evidenced by a decrease in the blood glucose level, maintenance of blood glucose level, or a slower increase in blood glucose level compared to patients that do not receive PALP. Therefore, in the treatment regimens in various embodiments of the present invention, the blood glucose levels of the mammal may be periodically monitored to determine a therapeutically effective amount to administer to the patient. Other methods of measuring the increased viability of islet cells, such as those discussed in the Examples, may also be used. Another factor to consider when determining the therapeutically effective amount is whether alkaline phosphatase and TF are used as part of a more complex regimen involving other treatments. For example, if a patient is simultaneously or alternatively treated with both PALP (with or without TF) and another therapy, the effective tolerated amount of the PALP may be less compared to a regimen when the subject is treated with PALP alone.

In another embodiment, the present invention provides a treatment regiment for treating a mammal with type 1 or type 2 diabetes by periodically administering a therapeutically effective amount of alkaline phosphatase or an active derivative thereof in the absence or presence of TF to the mammal. In these embodiments, the therapeutically effective amounts of PALP and TF may be administered once daily, twice or three times weekly, once a week or biweekly. Since the half-life time for PALP, the other alkaline phosphatases, and TF is relatively long and in the same range (5-7 days), the preparations of PALP and PALP plus TF may be administered twice a week or once a week.

In one embodiment, the islet cells, usually derived from cadavers, may be transplanted. It is known that after transplantation, vascular density in pancreatic islets is far from optimal [Mattson, G., Jansson, L. and Carlsson, P. O. (2002), “Decreased vascular density in mouse pancreatic islets after transplantation,” Diabetes, 51, 1362-1366]. TF is also known to promote migration of endothelial cells and increase vascular density [Carlevaro, M. F., Albini, A., Ribatti, D., Gentili, C., Benelli, R., Cermelli, S., Cancedda, R. and Cancedda, F. D. (1997), “Transferrin promotes endothelial cell migration and invasion: Implication in cartilage neovascularization,” J. Cell. Biol., 136, 1375-1384]. On the other hand, PALP may promote viability of endothelial cells as well. Thus, the TF- and PALP-containing compositions used in embodiments of the present invention may also enhance vascular density in islet transplants in addition to promoting islet cell survival as will be described in the Examples.

Embodiments of the invention also provide for the use of an alkaline phosphatase alone or in combination with TF or other promoters to enhance the viability of islets in diabetic patients who have not received islet transplantation. These patients may exhibit low islet function, manifested in greatly reduced insulin production and secretion. These patients require insulin treatments at regular intervals for their survival. The purpose of treatment of these patients with alkaline phosphatase and TF is to prevent further deterioration of their islets and concomitantly increase insulin secretion. Injectable forms of alkaline phosphatase and alkaline phosphatase plus TF, with or without other promoters of islet survival and insulin secretion, may be prepared and administered as described above.

In one embodiment, the present invention includes a method of reducing ROS-induced death of mammalian islet cells in vitro thereby maintaining or enhancing their viability, comprising contacting the islet cells with an effective amount of an alkaline phosphatase or an active derivative thereof This embodiment may be particularly useful in the isolation and transplantation of human islets [Lupi, R., Dotta, F., Marselli, L., Del Guerra, S., Masini, M., Santangelo, C., Patane, G., Boggi, U., Piro, S., Anello, M., Bergamini, E., Mosca, F., Di Mario, U., Del Prato, S. and Marchetti, P. (2002), “Prolonged exposure to free fatty acids has cytostatic and pro-apoptotic effects on human pancreatic islets,” Diabetes, 51, 1437-1442; Brandhorst, H., Brandhorst, D., Hesse, F., Ambrosius, D., Brendel, M., Kawakami, Y. and Bretzel, R. G. (2003), “Successful human islet isolation utilizing recombinant collagenase,” Diabetes, 52, 1143-1146].

In this embodiment, an alkaline phosphatase or alkaline phosphatase plus TF may be added to an isolation media used to isolate and store islet cells for the transplantation procedures. In the above cited and other published articles describing islet isolation, generally three different media are used: (i) Hanks balanced salt solution containing collagenase used for the digestion of pancreas, (ii) a solution containing 80% Histopaque and 20% Hanks balanced salt solution for the purification (centrifugation) procedure, and (iii) M199 culture medium in which the islets are suspended and maintained for a time period before transplantation. Other isolation media, however, may also be used.

In one embodiment, effective amounts of PALP, or another alkaline phosphatase, is added to each media for the entire duration of the isolation and maintenance procedures. Suitable concentrations of PALP used to supplement the isolation and growth media may depend on the purity of the PALP preparation. For example, if less purified commercial alkaline phosphatase is used, its concentration in the isolation and growth media may be in the range of about 15-200 μg/ml of media. If highly purified or homogeneous alkaline phosphatase is used, its recommended concentration may be in the range of about 1-15 μg/ml of media.

TF may also be added to the isolation and growth media in this embodiment. If TF is also used to supplement the isolation and growth media, the concentration of TF or it active derivative may be in the range of about 1-50 μg/ml of media. In addition to alkaline phosphatase and TF, any of the other promoters of islet survival described above may be added to any of the media used for the isolation and maintenance of islets. The cited articles provide guidance for their concentration in the isolation and growth media.

This invention may take on various modifications and alterations without departing from its spirit and scope thereof Accordingly, it is to be understood that this invention is not to be limited to the above described, but it is to be controlled by the limitations set forth in the following claims and any equivalents thereof It is also to be understood that this invention may be suitably practiced in the absence of any element not specifically disclosed herein.

In describing embodiments of the invention, specific terminology is used for the sake of clarity. The invention, however, is not intended to be limited to the specific terms so selected, and it is to be understood that each term so selected includes all technical equivalents that operate similarly.

### EXAMPLES

### Examples Relating to Methods

### Example 1

### Purification and Spectrophotometric Assay of PALP

Human PALP (Type XXIV, 1020 units of total activity) in a partially purified form was obtained commercially from Sigma-Aldrich. The steps for the isolation and purification of Sigma-Aldrich PALP product included homogenization of human placenta in Tris. This was followed by extraction of homogenate with butanol, exposure to heat (55° C.), three successive precipitations of protein with ammonium sulfate, re-suspension of protein, fractionation with ethanol, and Sephadex-G-200-gel filtration.

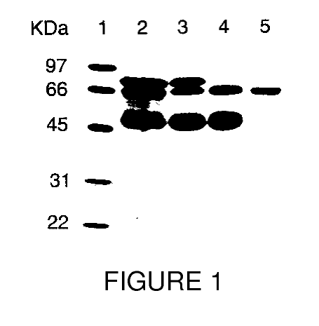

As determined by sodium dodecyl sulfate-polyacrylamide gel electrophoresis (SDS-PAGE), the partially purified PALP obtained from Sigma-Aldrich (denoted “commercial PALP” herein) was not homogeneous and contained other proteins. FIG. 1 shows a digital image of a gel separation of a preparation comprising commercial PALP without further purification, and other preparations of PALP of increasing purity. Separation of proteins was performed by conventional SDS-PAGE, and proteins were stained with coomassie blue stain. Lane 1 contains various molecular mass standards for comparison. Lane 2 represents a preparation containing commercial PALP with a strong 52 kDa band representing AT, another strong 66 kDa band representing a mixture of PALP and albumin, and an additional band representing transferrin. Lanes 3 and 4 represent preparations comprising commercial PALP material after further purification steps (described below), and lane 5 represents a preparation of homogeneous PALP obtained by the complete purification procedure described below. As mentioned earlier, the commercial PALP preparation (shown in lane 2) contains about 10% PALP, 12% TF, 30% α₁-antitrypsin (AT), 35% albumin, and 13% TF derivatives (proteolytic degradation products of TF).

A purification procedure consisting of several steps and described in She, Q. -B., Mukherjee, J. J., Huang, J. -S., Crilly, K. S. and Kiss, Z. (2000), “Growth factor-like effects of placental alkaline phosphatase in human and mouse embryo fibroblasts,” FEBS Lett., 469, 163-167 was performed to further purify the commercially obtained PALP to homogeneity.

The solution of commercial PALP was prepared by dissolving 350 mg of commercial PALP into 10 ml of buffer A (0.1 M sodium acetate, 0.5 M NaCl, 1 mM MgCl₂, 1 mM CaCl₂, adjusted to pH 6.5). This solution was then further purified by successive Concanavalin A-Sepharose, Q-Sepharose and t-butyl hydrophobic interaction chromatography.

The solution was passed through a Concanavalin A-Sepharose column followed by an elution step using buffer A as solvent. For elution, buffer A, composed of 50 mM α-methyl-D-mannopyranoside, was used. The active fractions collected from the effluent were pooled and dialyzed against buffer B (50 mM Tris-HCL at pH 7.7). SDS-PAGE separation of the collected and dialyzed fraction is shown in FIG. 1 in lane 3. It should be mentioned that a different fraction from the Concanavalin A-Sepharose column could be collected which contains only albumin (not shown here). Measurement of the weight of dried albumin fraction revealed that it represents about 35% of the total original protein content.

The collected and dialyzed fraction from the previous step was then passed through a Q-Sepharose column. The fraction of interest was eluted with buffer B using a linear gradient of 0-250 mM potassium phosphate at a pH of 7.5. The active fractions from the Q-Sepharose column were pooled and dialyzed against phosphate-buffered saline and concentrated by Amicon ultrafiltration. SDS-PAGE separation of the collected and dialyzed fraction is shown in FIG. 1 in lane 4, which demonstrates that at least two major proteins are still present in the fraction after dialysis. Of interest to note here is that a different fraction from the Q-Sepharose column could be collected which contains only TF (not shown here). Measurement of the weight of dried TF fraction revealed that it represents about 12% of total protein content.

Then, the collected and dialyzed fraction from the previous step was purified to homogeneity by t-butyl hydrophobic interaction chromatography (HIC). Prior to adding the fraction to the t-butyl HIC column, the fraction was made 2 M in ammonium sulfate, and the pH was adjusted to 6.8. The 5-ml bed volume t-butyl HIC cartridge (BIO-RAD, Hercules, Calif.) was connected to a fast performance liquid chromatography (FPLC) system from PHARMACIA (Peapack, N.J.). The fraction was introduced to the HIC column, and the column was eluted with buffer C (100 mM sodium phosphate buffer, 2 M ammonium sulfate at pH 6.8). The column was eluted with buffer C until a first protein-containing fraction completely eluted, and then a negative gradient of 2 M-to-0 M ammonium sulfate in 100 mM sodium phosphate at pH 6.8 was passed over the column. The negative linear gradient was used to elute a second protein-containing fraction, which contained the enzymatically active PALP protein. A different fraction contained α₁-antitrypsin (not shown here). Measurement of the weight of dried α₁-antitrypsin fraction revealed that it represents about 30% of the total protein content.

The enzymatically active PALP fraction from the HIC separation was dialyzed against phosphate buffered saline and concentrated by Amicon ultrafiltration. The presence and purity of the PALP enzyme in the fraction was confirmed by SDS-PAGE. After electrophoretic separation, the gel was stained using coomassie blue or silver stain for visual observation of protein bands. A single protein band was observed with an approximate molecular weight of 66 kDa. The pure PALP was further identified by sequence analysis performed by the Mayo Clinic Protein Core Facility (Rochester, Minn., US). Measurement of the weight of dried PALP fraction revealed that it represents about 10% of the total protein content.

PALP enzyme activity was assayed using a spectroscopic method by monitoring the hydrolysis of 4-nitrophenylphosphate (as an increase in absorbance at 410 nm) at room temperature (22° C.) as described in Chang, G. -G., Shiao, M. -S., Lee, K. -R. and Wu, J. -J. (1990), “Modification of human placental alkaline phosphatase by periodate-oxidized 1,N⁶-ethenoadenosine monophosphate,” Biochem. J., 272, 683-690. Activity analysis of 5-10 μg purified enzyme was performed in 1 mL incubation volume containing 50 mM Na₂CO₃/NaHCO₃, 10 mM MgCl₂, 10 mM 4-nitrophenylphosphate at pH 9.8. The extinction coefficient of 4-nitrophenol was taken as 1.62×10⁴ M⁻¹ cm⁻¹. An enzyme activity of 1 U (unit) is defined as 1 μmol substrate hydrolyzed/min at 22° C. at pH 9.8.

### Example 2

### Use of the MTT Assay to Determine Cell Viability

In the Examples below, an MTT assay was used to determine the relative number of viable cells after treatments. This colorimetric assay is based on the ability of living cells, but not dead cells, to reduce 3-(4, 5-dimethyl thiazol-2-yl)-2, 5-diphenyltetrazolium bromide. [Carmichael, J, De Graff, W. G., Gazdar, A. F., Minna, J. D. and Mitchell, J. B. (1987), “Evaluation of tetrazolium-based semiautomated colorimetric assay: Assessment of chemosensitivity testing,” Cancer Res., 47, 936-942], which is incorporated by reference herein. For this assay, cells were plated in 96-well plates, and the MTT assay was performed as described in the above article both in untreated and treated cell cultures. The MTT assay also was performed at the time when the treatment was started to allow for assessment of the proliferation and survival rates in the control and treated cell cultures. Absorption was measured at wavelength=540, indicated in the Tables below as A₅₄₀. In the MTT assay, higher values mean proportionally higher numbers of viable cells.

### Example 3

### Determination of Relative Number of Dead RIN Cells by Cell Cycle Analysis

The use of the rat clonal β-cell RIN 1046-38 line to determine fatty acid-induced β-cell apoptosis has been published [Eitel, K., Staiger, H., Brendel, M. D., Brandhorst, D., Bretzel, R. G., Haring, H. U. and Kellerer, M. (2002), “Different role of saturated and unsaturated fatty acids in β-cell apoptosis,” Biochim. Biophys. Res. Commun., 299, 853-856]. The cells were maintained in 199-Earle's salts medium (M199 medium) containing 10% fetal calf serum and 5 mM glucose under an atmosphere of 93% air and 7% CO₂, at 37° C. The cells, used between passages 18-28, were split once a week using 0.1% trypsin-EDTA solution. For the experiments, the cells were seeded into 12-well plates in serum-containing medium. After 24 hours, the medium was replaced with serum-free medium followed by treatments with commercial PALP for 48 hours. At the end of the incubations, detached cells were harvested from the supernatant by centrifugation and added to non-detached cells harvested by trypsinization. Cells were washed with phosphate-buffered saline (PBS), fixed in 70% ice-cold ethanol, centrifuged, and washed with PBS. After staining with propidium iodide (50 μg/ml) diluted in PBS containing RNase (100 μg/ml), cells were subjected to flow cytometric analysis of DNA content using a Becton-Dickinson FACScalibur cytometer. Percentages of cells in the different cell cycle phases were calculated by CellQuest software (Becton-Dickinson, Heidelberg, Germany). This program generates a flow cytometry histogram indicating the percentage of cells in the various phases of cell cycle. Dead cells are detected as the sub-G1 fraction. All steps involved in this procedure are known.

### Example 4

### Determination of Fatty Acid-induced Death of Human Islet Cells

Human islets were isolated from the pancreas of multiorgan donors using a known method [Lupi, R., Dotta, F., Marselli, L., Del Guerra, S., Masini, M., Santangelo, C., Patane, G., Boggi, U., Piro, S., Anello, M., Bergamini, E., Mosca, F., Di Mario, U., Del Prato, S. and Marchetti, P. (2002), “Prolonged exposure to free fatty acids has cytostatic and pro-apoptotic effects on human pancreatic islets,” Diabetes, 51, 1437-1442]. The procedure to determine the number of human islet cells that died due to fatty acid exposure was performed by following a method reported by Eitel et al. [Eitel, K., Staiger, H., Brendel, M. D., Brandhorst, D., Bretzel, R. G., Haring, H. U. and Kellerer, M. (2002), “Different role of saturated and unsaturated fatty acids in β-cell apoptosis,” Biochim. Biophys. Res. Commun., 299, 853-856], which report is hereby incorporated by reference. Two days after plating the human islets, cells were trypsinized and seeded on collagen A (Biochrom)-coated glass coverslips. Two days after seeding on coverslips, the medium was exchanged for medium containing 0.5-1 mM fatty acid and 2.5 % bovine serum albumin. The serum optionally contained 5 mM glucose, highly purified 15 μg/ml PALP and, if included, commercial human 15 μg/ml TF. After treatments for 72 hours, dUTP nick-end labeling (TUNEL) assay was performed according to the manufacturer's instructions for the in situ cell death detection kit (Boehringer-Mannheim, Mannheim, Germany).

### Examples Illustrating that PALP and TF Enhance the Survival of Rodent and Human Islet β-cells

### Example 5

### Prevention of Serum-free Medium-induced Death of NIT-1 Mouse Islet Cells by PALP and TF

NIT-1 β-cells were obtained from American Type Culture Collection (ATCC CRL-2055). These cells were originally isolated from transgenic NOD mouse carrying SV 40 large T antigen gene on a rat insulin promoter. NIT-1 cells exhibit the ultrastructural features typical of differentiated β-cells, but upon prolonged cultivation they can spontaneously develop beta adenomas. Practically all cells in this cell population contain and secrete insulin, while at the same time they retained their ability to proliferate in the presence of an appropriate stimulus. The cells, maintained in Ham's F12K medium containing 10% heat-inactivated dialyzed fetal bovine serum, were used between passages 25-28.

Cells were seeded into 96-well plates at 8,000 cells/well in 10% serum-containing medium. After 24 hours, the medium was changed for serum-free medium, followed (within 2-3 hours) by treatments with commercial PALP (cPALP; purchased from Sigma-Aldrich), highly purified PALP (hpPALP), partially iron-saturated commercial TF (catalog number: T 3309 according to the 2004/2005 Sigma-Aldrich Catalog), or commercial anti-PALP antibody (PALP-AB) (catalog number: A 2951 according to the 2004/2005 Sigma-Aldrich Catalog). Each protein stock solution was made up in Ham's F12K medium and added in 10-μl volume to the incubation medium (final volume: 110-μl). Treatments and the final concentrations of proteins are shown in TABLE 1. Treatments were performed for 72 hours, followed by the MTT assay to determine the relative number of viable cells (absorption being measured at wavelength=540; indicated in Table 1 as A₅₄₀); higher numbers mean proportionally higher numbers of viable cells. The mean values±std. dev. of 8 determinations (in 8 separate wells) for each treatment is shown in TABLE 1. The MTT assay was also performed on the same day when the treatments were started (0 hour).

The results indicate that most islet cells died after incubating them in serum-free medium for 72 hours. However, in cell cultures treated with commercial PALP cell death was prevented and the number of viable cells actually increased. This indicates that the commercial PALP not only prevented cell death but also increased cell proliferation. Increased cell proliferation is possible, because these cells are undifferentiated in contrast to those islet cells that are derived from normal human islets. Highly purified PALP (hpPALP) was less effective than commercial PALP even at a concentration (15 μg/ml) which is higher than the amount of PALP present in the commercial PALP (it is estimated that PALP represents only about 10% of the total content of commercial PALP preparation). However, optimal concentrations of purified PALP and TF in combination were as effective as commercial PALP both in preventing β-cell death and increasing cell numbers. Clearly, TF could not entirely account for the combined effects of PALP and TF, because in that case the combined effects would be independent of the PALP concentration. As shown in TABLE 1, the combined effects of PALP and TF increased by increasing the concentration of PALP. The results also reveal that the anti-PALP antibody only partially inhibited the effect of commercial PALP.

TABLE 1
Effects of PALP in the absence or presence of TF on the survival of
NIT-1 cells in serum-free medium.

| Additions | A₅₄₀ |
|---|---|
| None, 0 hr | 1,214 ± 0.090 |
| None, 72 hrs | 0.297 ± 0.069 |
| cPALP, 50 μg/ml; 72 hrs | 1,636 ± 0.082 |
| hpPALP, 2.5 μg/ml; 72 hrs | 0.521 ± 0.033 |
| hpPALP, 5 μg/ml; 72 hrs | 0.928 ± 0.076 |
| hpPALP, 15 μg/ml; 72 hrs | 1,328 ± 0.065 |
| hpPALP, 2.5 μg/ml + TF, 20 μg/ml; 72 hrs | 1,123 ± 0.05 1 |
| hpPALP, 5 μg/ml + TF, 20 μg/ml; 72 hrs | 1,479 ± 0.040 |
| hpPALP, 15 μg/ml + TF, 20 μg/ml; 72 hrs | 1,694 ± 0.093 |
| cPALP, 50 μg/ml + PALP-AB, 100 μg/ml; 72 hrs | 0.665 ± 0.130 |

### Example 6

### Prevention of STZ-induced Death of NIT-1 Mouse Islet Cells by PALP

Although serum-free medium is known to enhance the production of ROS, the data presented in TABLE 1 left open the possibility that PALP and TF simply substituted for growth factors and did not act by preventing ROS formation. To further clarify the role of ROS in islet β-cell death, streptozotocin (STZ) was used in Example 2 to increase ROS formation in NIT-1 cell cultures. Others have shown that STZ causes islet β-cell damage via the release of ROS and that antioxidants can prevent such damage [Chen, H., Carlson, E. C., Pellet, L., Moritz, J. T. and Epstein, P. N. (2001), “Overexpression of metallothionein in pancreatic β-cells reduces streptozotocin-induced DNA damage and diabetes,” Diabetes, 50, 2040-2046].

In this example NIT-1 cells were cultured and seeded into 96-well plates as described for TABLE 1. After 24 hours, the medium was replaced with fresh 2% bovine serum albumin-containing medium, followed by treatments with STZ and highly purified PALP. Treatments were for 24 hours followed by the MTT assay to determine the relative number of viable cells (expressed as A₅₄₀). The mean value±std. dev. of 8 determinations (in 8 separate wells) for each treatment is shown in Table 2.

Over a 24 hours incubation period, STZ reduced the number of viable cells by about 28%. As shown in Table 2, a lower concentration of highly purified PALP (5 μg/ml) partially prevented the STZ-induced death of islet cells, while a higher concentration (15 μg/ml) of PALP provided full protection against STZ-induced death of the islet cells. These data indicate that PALP may prevent or reduce ROS-induced islet β-cell death in vivo.

TABLE 2
PALP prevents STZ-induced islet cell death.

| Additions | A₅₄₀ |
|---|---|
| None | 1,694 ± 0.083 |
| STZ, 20 mM | 1,224 ± 0.055 |
| STZ + PALP, 5 μg/ml | 1,541 ± 0.071 |
| STZ + PALP, 15 μg/ml | 1,673 ± 0.074 |

### Example 7

### Partial Prevention of Serum-free Medium-induced Death of RIN 1046-38 Cells by PALP

RIN 1046-38 cells were selected from RIN-m islet tumor cells that are available from American Type Culture Collection (ATCC CRL 2057). They are often used cell models for the study of molecular and cellular events in β-cells. Cells were seeded into 12-well plates in 10% serum-containing medium. When the cultures reached 85-90% confluency, the medium was changed to serum-free medium, followed by the addition of highly purified PALP. The incubations continued for 48 hours. The percentage of dead cells, presented as SubG1 cells, was determined by cell cycle analysis as described above. For each treatment, 3 cultures were analyzed. The data, shown in TABLE 3, are expressed as mean values±std. dev. of 3 determinations. The results indicate that in the absence of serum, the percentage of dead cells increased by about 12%. PALP at 5 μg/ml concentration substantially inhibited cell death with a maximum effect achieved at 10 μg/ml concentration of the protein.

TABLE 3
PALP partially protects RIN 1046-38 cells
from serum-free medium-induced death.

| | % of SubG1 |
|---|---|
| Addition | (dead) cells (total = 100%) |
| 10% serum | 6.1 ± 1.4 |
| Serum-free medium | 18.9 ± 1.5 |
| Serum-free medium + PALP, 5 μg/ml | 11.9 ± 3.4 |
| Serum-free medium + PALP, 10 μg/ml | 10.0 ± 1.5 |
| Serum-free medium + PALP, 40 μg/ml | 10.2 ± 1.9 |

### Example 8

### Protective Effect of PALP Against Fatty Acid-induced Death of RIN 1046-38 Cells

It has been well established that saturated fatty acids (palmitic acid or stearic acid), but not unsaturated fatty acids, induce apoptotic cell death of RIN 1046-38 cells [Eitel, K., Staiger, H., Rieger, J., Mischak, H., Brandhorst, H., Brendel, M. D., Bretzel, R. G., Haring, H. U. and Kellerer, M. (2003), “Protein kinase C δ activation and translocation to the nucleus are required for fatty acid-induced apoptosis of insulin-secreting cells,” Diabetes, 52, 991-997]. Since fatty acids are considered to contribute to β-cell loss in vivo, protection of these cells against fatty acid-induced death can improve the condition of diabetic subjects.

RIN 1046-38 cells were used to examine if PALP can protect against fatty acid-induced cell death. Palmitic acid and stearic acid were purchased from Sigma-Aldrich. Stock solutions of fatty acids were prepared as follows. Fatty acids were dissolved in 200 mM ethanol and then diluted 1:25 with Krebs-Ringer-Hepes buffer containing 20% bovine serum albumin (fraction V, fatty acid-free; from Sigma-Aldrich). The fatty acid mixtures were gently agitated at 37° C. under nitrogen overnight. The cells were seeded into 96-well plates at a density of 8,000 cells per well. When the cultures reached ˜70% confluence, fatty acid or a corresponding amount of albumin and highly purified PALP (suspended in the medium) were added to the medium and incubations continued for 24 hours. This was followed by the MTT assay to determine the relative number of viable cells. The data, shown in Table 4, are expressed as mean values±std. dev. of 8 determinations.

As shown in TABLE 4, both palmitic acid and stearic acid decreased the number of viable cells by 25-30% compared to the untreated incubated control. PALP alone did not significantly change the number of viable cells but it completely prevented the death of RIN cells induced by both fatty acids.

TABLE 4
PALP prevents fatty acid-induced death of RIN 1046-38 cells.

| Additions | A₅₄₀ |
|---|---|
| None | 1,602 ± 0.121 |
| PALP, 15 μg/ml | 1,712 ± 0.091 |
| Palmitic acid, 1 mM | 1,130 ± 0.069 |
| Palmitic acid, 1 mM + PALP, 15 μg/ml | 1,672 ± 0.112 |
| Stearic acid, 1 mM | 1,206 ± 0.063 |
| Stearic acid, 1 mM + PALP, 15 μg/ml | 1,619 ± 0.086 |

### Example 9

### Prevention of Fatty Acid-induced Death of Human Islet Cells by PALP

The procedure used for this experiment is described above. The fatty acid stock solutions were made as described in Example 4. The inhibitory effects of saturated fatty acids and the stimulatory effects of highly purified PALP on the viability of human islet cells are shown in TABLE 5. The values represent the average of two determinations; both measured values are given in parenthesis.

The results show that human islet cells, in contrast to RIN cells, are less sensitive to palmitic acid. Accordingly, PALP had no clear conclusive effects on cell viability. At both the 0.5 and 1 mM concentrations, palmitic acid only slightly increased the number of dead cells and PALP appeared to have protective effects only at the 0.5 mM concentration. It should be noted, however, that the 0.5 mM concentration is closer to the physiological concentration of free fatty acids under pathological conditions than the 1 mM concentration. In contrast to palmitic acid, stearic acid was shown to induce the death of human islet β-cells, particularly at 1 mM concentration. Moreover, as shown in TABLE 5, PALP provided significant protection against stearic acid-induced cell death. These data indicate PALP can at least partially protect human islet β-cells against death induced by physiological concentrations of free fatty acids.

TABLE 5
PALP partially protects human islet β-cells
against fatty acid-induced death.

| | % of dead cells |
|---|---|
| Additions | (total = 100%)* |
| None | 1.9 (1.5, 2.4) |
| PALP, 15 μg/ml | 1.4 (0.8, 2.0) |
| Stearic acid, 1 mM | 26.2 (24.3, 28.1) |
| Stearic acid, 1 mM + PALP, 15 μg/ml | 16.9 (14.1, 19.7) |
| Palmitic acid, 1 mM | 5.0 (4.3, 5.7) |
| Palmitic acid, 1 mM + PALP, 15 μg/ml | 4.7 (3.9, 5.5) |
| Stearic acid, 0.5 mM | 12.2 (11.3, 13.1) |
| Stearic acid, 0.5 mM + PALP, 15 μg/ml | 8.1 (8.8, 7.4) |
| Palmitic acid, 0.5 mM | 4.8 (4.1, 5.5) |
| Palmitic acid, 0.5 mM + PALP, 15 μg/ml | 2.5 (1.9, 3.1) |

*Numbers in parenthesis indicate the actual measured values.

### Example 10

### PALP Inhibits Proliferation of T47D Breast Cancer Cells

The purpose of this example was to determine if the stimulatory effects of PALP on cell viability can be demonstrated in several different other cell types.

All cell lines were obtained from American Type Culture Collection. Generally, cells were seeded into both 12-well and 96-well plates in a medium containing 10% serum. After the cultures became confluent, the medium was replaced with serum-free medium and the cells were incubated further for 3-6 days in the absence or presence of 5-15 μg/ml of highly purified PALP. Following incubation, digital images of the 12-well plates were taken and an MTT assay was performed on the cells in the 96-well plates.

The first cell line tested for the effect of PALP was the T47D human breast cancer cell line (ATCC HTB 133) maintained in RPMI medium containing 0.2 IU bovine insulin/ml and 10% fetal bovine serum. After reaching confluence in 10% serum-containing medium, the cells were incubated in serum-free medium for 6 days in the absence or presence of 5 or 15 μg/ml of highly purified PALP. Before the digital images were taken, the cells were quickly washed to remove most of the dead cells. The digital images are shown in FIGS. 2 A-D. The initially confluent cell cultures (FIG. 2A) became about 80% confluent when incubations in serum-free medium were performed for 6 days in the absence of PALP (FIG. 2B). In the presence of 5 μg/ml of PALP (FIG. 2C) or 15 μg/ml of PALP (FIG. 2D) the cultures lost even more cells and became about 65% and 50% confluent, respectively. Parallel determination of cell numbers with the MTT assay confirmed that PALP induced the loss of 15-30% of cells compared to the untreated control cell cultures.

In similar experiments, PALP failed to enhance survival (in serum-free medium) of 3A-sub E cells derived from human placenta (ATCC CRL 1584), Caov-3 human adenocarcinoma cells derived from the ovary (ATCC HTB 75), and Hep G2 human hepatocellular carcinoma cells (ATCC HB 8065).

### Example 11

### Stimulatory Effect of PALP on the Secretion of Insulin from Human Islets

Human islets were isolated from the pancreas of multiorgan donors using a known method [Lupi, R., Dotta, F., Marselli, L., Del Guerra, S., Masini, M., Santangelo, C., Patane, G., Boggi, U., Piro, S., Anello, M., Bergamini, E., Mosca, F., Di Mario, U., Del Prato, S. and Marchetti, P. (2002), “Prolonged exposure to free fatty acids has cytostatic and pro-apoptotic effects on human pancreatic islets,” Diabetes, 51, 1437-1442]. Six groups of islets were cultured in 60-mm Petri dishes (50 islets of similar size per dish; 2 dishes for each group) in CMRL 1066 medium supplemented with 5 mM glucose, 0.05 mM dithiothreitol, 2 mM glutamine, 1 mM Na-pyruvate, 20 μg/ml Ciprobay, 100 IU/ml penicillin, 100 μg/ml streptomycin, 10 mM HEPES, 0.01 mM hydrocortisone, and 10% fetal calf serum. After about 4 days, the medium was replaced with fresh medium. The fresh medium contained 2.8 mM glucose, or 10 mM glucose, or 22 mM glucose. For each concentration of glucose, one group received medium containing 50 μg/ml of commercial PALP (described above) in addition to the glucose. The other group received only medium but no PALP. The islets were then incubated for 60 minutes and the amount of insulin secreted by the islets was determined with a radioimmunoassay kit purchased from Pharmacia Diagnostics AB (Uppsala, Sweden). The results, presented in TABLE 6, are the average of two measured values. For each group, the measured values differed by less than 15%.

While the results clearly show that the addition of commercial PALP enhanced insulin release by the islets at different glucose concentrations, presently it is not known whether such increased insulin release merely reflected increased viability of islet cells, or was a genuine increase in insulin secretion, or both.

TABLE 6
Commercial PALP stimulates insulin release from human islets.

| | Secreted insulin (ng/μl medium) | | |
|---|---|---|---|
| Addition | 2.8 mM Glucose | 10 mM Glucose | 22 mM Glucose |
| None | 10.2 | 12.3 | 22.3 |
| PALP, | 38.6 | 38.8 | 52.1 |
| 50 μg/ml | | | |

### Example 12

### Enhancing the Secretion of Insulin from Human Islets with PALP and TF

In this example, islets were treated with highly purified PALP alone as well as highly purified PALP and TF. Islets were isolated and prepared in the same manner as in Example 1 except that in this example, nine groups of islets were prepared. The fresh medium contained 2.8 mM glucose, or 10 mM glucose, or 22 mM glucose. For each concentration of glucose, one group received medium with 15 μg/ml of highly purified PALP in addition to the glucose. Another group received medium with 15 μg/ml of highly purified PALP plus 15 μg/ml of partially iron-saturated TF (catalog number: T 3309 according to the 2004/2005 Sigma-Aldrich Catalog). The islets were then incubated for 60 minutes and the amount of insulin secreted by the islets was determined with a radioimmunoassay kit purchased from Pharmacia Diagnostics AB (Uppsala, Sweden). The results, presented in TABLE 7, are the average of two measured values. For each group, the measured values differed by less than 15%.

The results show that, like in the previous experiment, glucose challenge enhanced insulin release indicating that purified PALP enhanced insulin release at each glucose concentration. Furthermore, it can be seen that the addition of TF enhanced the effects of PALP on the production and secretion of insulin by the islet cells. Comparison of data in TABLE 6 and TABLE 7 indicates that highly purified PALP alone was somewhat less effective than commercial PALP. However, in the presence of TF, the highly purified PALP approximated the effectiveness of commercial PALP. Again, it is not clear whether increased insulin release merely reflected increased viability of islet cells, or was a genuine increase in insulin secretion, or both.

TABLE 7
Highly purified PALP and TF stimulate insulin release from human islets.

| | Secreted insulin (ng/μl medium) | | |
|---|---|---|---|
| Addition | 2.8 mM Glucose | 10 mM Glucose | 22 mM Glucose |
| None | 9.1 | 14.7 | 29.6 |
| PALP, | 28.4 | 30.2 | 47.9 |
| 15 μg/ml | | | |
| PALP, | 35.8 | 40.4 | 57.3 |
| 15 μg/ml + | | | |
| TF, 15 μg/ml | | | |

### Example 13

### Injection of a 1:1 Mixture of Purified PALP and TF Results in Increased Blood Vessel Formation in the Islets

In this experiment C57/Black female mice, weighing 22-23 g, were used. Five mice were untreated, while 5 mice were intraperitoneally injected 48 mg/kg of a 1:1 mixture of highly purified PALP and partially iron-saturated TF on days 0, 2, 4, and 6. Samples were taken from the pancreas on day 8. The excised tissue samples were fixed in 4% paraformaldehyde in phosphate-buffered saline and embedded in paraffin so that several consecutive cross-sections could be made. Sections (5-6 μm) were stained with hematoxylin/eosin (“H&E”) with a standard procedure well known in the art. For both the untreated and treated groups of animals ten sections were evaluated for the presence of islets and the presence of blood vessels in the islets. On average, one section contained about 2 islets. Blood-containing blood vessels are stained red, which ensures their straightforward recognition. Although this method underestimates the total number of vessels compared to the use of a more specific immunostaining methods, it provides a good estimate of the relative numbers because the error should be about the same in both groups. In the samples derived from untreated mice, one islet contained on average 2.4 morphologically distinct blood vessels while treatment with PALP raised this number to 4.6. Both the number of islets studied (about 20 islets in each group) and the difference between the two groups is sufficiently large to conclude that injection of a 1:1 mixture of PALP and TF enhanced the number of blood vessels in the islets. More blood vessels improve nutrient supply inside the islets that should result in increased viability of islet cells.

1. A method of reducing or preventing death and thereby maintaining or enhancing viability and function of mammalian islet cells, comprising administering an effective amount of an alkaline phosphatase or an active derivative thereof to islet cells.

2. The method of claim 1 wherein the alkaline phosphatase or active derivative is administered to islet cells in vivo.

3. The method of claim 1 wherein the alkaline phosphatase or active derivative is administered to islet cells in vitro.

4. The method of claim 1, wherein the islet cells are also administered an effective amount of transferrin or an active derivative.

5. The method of claim 4, wherein the islet cells are also administered a promoter of islet cell viability.

6. The method of claim 1, wherein the islet cells are transplanted into a mammal.

7. The method of claim 1, wherein alkaline phosphatase is present in media used to isolate or store islet cells prior to transplantation.

8. The method of claim 7, wherein the concentration of less purified and homogeneous alkaline phosphatase in the media is in the range of 15-200 μg/ml and 1-15 μg/ml, respectively.

9. The method of claim 8, further comprising transferrin, or an active derivative thereof, at 1 to 50 μg/ml concentration.

10. The method of claim 7, further comprising one or more promoters of islet viability.

11. The method of claim 1, wherein the alkaline phosphatase is administered via injection chosen from intravenous, intraperitoneal, subcutaneous, intraarterial, intradermal, intraportal, intrapancreas, or intramuscular delivery routes.

12. The method of claim 1, wherein the amount of alkaline phosphatase is between 0.01 to about 2.5 gram per square meter of calculated surface area for a mammal.

13. The method of claim 1, wherein the amount of alkaline phosphatase is between 0.1 to about 1.0 gram per square meter of calculated surface area for the mammal.

14. The use of alkaline phosphatase and transferrin, or active derivatives thereof, in the manufacture of a composition useful for the enhancement of viability and function of islet cells in vivo.

15. A medicament for treating Type 1 and Type 2 diabetes by maintaining or enhancing viability of islet cells comprising alkaline phosphatase and transferrin or active derivatives thereof.

16. A composition for preventing or reducing death of mammalian islet cells and thereby preserving and enhancing viability and insulin secretion comprising about 0.01-2.5 gram per square meter of surface area of a mammal of alkaline phosphatase and transferrin.
